(19) United States
(12) Patent Application Publication
(10) Pub. No.: US 2009/0276020 A1
(43) Pub. Date: Nov. 5, 2009

(54) TOOLS FOR DELIVERING IMPLANTABLE MEDICAL LEADS AND METHODS OF USING AND MANUFACTURING SUCH TOOLS

(75) Inventors: Elizabeth Nee, Chicago, IL (US); Thao Thu Nguyen, South Bloomington, MN (US); Scott Salys, Los Angeles, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/114,616

(22) Filed: May 2, 2008

(51) Int. Cl. A61N 1/05

(52) U.S. Cl. 607/116

(57) ABSTRACT

Disclosed herein is a tool for implanting a medical lead. In one embodiment, the tool includes a body, an electrode, and a conductor. The body includes a distal end and a proximal end. The electrode is supported by the body. The conductor is in electrical contact with the electrode and extends along the body from the electrode to the proximal end. The electrode and conductor form an electrically conductive path that extends from a surface of the electrode to a proximal most point of the conductor on the body. The electrical resistance of the electrically conductive path is at least approximately 100 Ohms.

### FIELD OF THE INVENTION

The present invention relates to medical devices designed to operate with navigation and visualization systems. More specifically, the present invention relates to tools for delivering implantable medical leads, wherein the tools are designed to be tracked via navigation and visualization systems.

### BACKGROUND OF THE INVENTION

Currently, physicians use fluoroscopy for navigation and guidance when implanting leads for pacing, defibrillation, or cardiac resynchronization therapy (“CRT”). Fluoroscopy has some significant drawbacks. For example, fluoroscopy exposes the patient and medical staff to radiation, and special clothing and equipment is needed in an attempt to protect against the radiation. Also, fluoroscopy equipment is expensive. Finally, the images provided by fluoroscopy are often less than desirable.

Navigation and imaging systems such as the St. Jude Medical, Inc. Ensite Array™ multi-electrode array catheter system and Ensite NavX™ system allow visualization and tracking of electrode equipped medical devices, such as electrophysiology (“EP”) catheters, within a patient without employing fluoroscopy. In order to ensure adequate signal emanation and detection to perform the primary sensing and/or treatment purposes of an EP catheter, pacing lead, or other electrode equipped medical device, material conductivity and component connections are critical to the design of such devices and their electrodes. Such electrodes and their electrical connections are expensive to manufacture. As a result, providing such electrodes to a lead delivery tool, such as an introducer sheath, catheter, etc., simply for the purposes of visualization and tracking the delivery tool via a non-fluoroscopy visualization and tracking system is unnecessarily expensive.

There is a need in the art for a delivery tool usable with a non-fluoroscopy visualization and tracking system that is cost effective to manufacture. There is also a need in the art for methods of using and manufacturing such a tool.

### SUMMARY

Disclosed herein is a tool for implanting a medical lead. In one embodiment, the tool includes a body, an electrode, and a conductor. The body includes a distal end and a proximal end. The electrode is supported by the body. The conductor is in electrical contact with the electrode and extends along the body from the electrode to the proximal end. The electrode and conductor form an electrically conductive path that extends from a surface of the electrode to a proximal most point of the conductor on the body. The electrical resistance of the electrically conductive path is at least approximately 100 Ohms.

Disclosed herein is a tool for implanting a medical lead. In one embodiment, the tool includes a distal end, a proximal end, a first layer, a conductor, a second layer and an electrode. The conductor extends along an outer surface of the first layer. The second layer extends over the outer surface of the first layer. The electrode extends over the outer surface of the first layer, forms a portion of the second layer and is in electrical contact with an electrically conductive portion of the conductor.

Disclosed herein is a tool for implanting a medical lead. In one embodiment, the tool includes a distal end, a proximal end, a first layer, a conductor, a second layer, and an electrode. The conductor forms a portion of the first layer. The second layer extends over the outer surface of the first layer. The electrode extends over the outer surface of the first layer, forms a portion of the second layer and is in electrical contact with the conductor.

Disclosed herein is a tool for implanting a medical lead. In one embodiment, the tool includes a distal end, a proximal end, a first layer, a conductor, and an electrode. The conductor extends along the surface of the first layer. The electrode extends over the outer surface of the first layer and is in electrical contact with the conductor. The conductor and/or the electrode are formed of an electrically conductive ink.

Disclosed herein is a system for implanting a medical lead. In one embodiment, the system includes an imaging system (e.g., an Ensite™ system as manufactured by St. Jude Medical, Inc.) and a tool for delivering a medical lead. The imaging system includes a power and imaging device and surface electrode pairs electrically coupled to the device. The imaging system generates generally orthogonal electric fields via the electrodes pairs. The tool includes a tubular body having a conductor extending from a proximal end of the body to an electrode supported on the body. The conductor is electrically coupled at the proximal end of the body to the device. The electrode is visible via the imaging system but generally inadequate for sensing or treatment purposes due to the high electrical resistance of an electrically conductive path extending from a surface of the electrode to a proximal most point of the conductor on the body.

Disclosed herein is a method of delivering an implantable medical lead. In one embodiment, the method includes: electrically coupling a tool to an imaging system (e.g., an Ensite™ system as manufactured by St. Jude Medical, Inc.); generating generally orthogonal electric fields in a patient with the imaging system; tracking the tool to a lead implantation site, wherein the tool includes an electrode that is visible within the patient via the imaging system, but the electrode is generally inadequate for sensing or treatment purposes due to the high electrical resistance of an electrically conductive path extending from a surface of the electrode to a proximal most point of the conductor on the body; and delivering the lead to the implantation site through the tool.

Disclosed herein is a method of manufacturing a tool for delivering an implantable medical lead. In one embodiment, the method includes: providing a inner tubular layer, extending an jacketed conductor along a surface of the inner tubular layer; exposing a conductive core of the jacketed conductor along a region of the inner tubular layer; providing an outer tubular layer over the inner tubular layer and jacketed conductor, wherein an electrode region of the outer tubular layer is impregnated with an electrically conductive material; aligning the electrode region with the region of the inner tubular layer corresponding to the exposed conductive core; and causing the outer tubular layer to adhere to the inner tubular layer.

Disclosed herein is a method of manufacturing a tool for delivering an implantable medical lead. In one embodiment, the method includes: providing a inner tubular layer including a conductor region forming a portion of the inner tubular layer, wherein the conductor region is impregnated with an electrically conductive material; and providing an electrode in electrical communication with the conductor region.

Disclosed herein is a method of manufacturing a tool for delivering an implantable medical lead. In one embodiment, the method includes: providing a tubular layer; supporting a conductor on the tubular layer; and providing an electrode in electrical communication with the conductor, wherein at least one of the conductor or electrode is an electrically conductive ink.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a diagram illustrating a mapping system being employed on a patient.

FIG. 2 is an isometric view of the tool.

FIG. 3 is a longitudinal elevation of the tool with the electrodes and an outer layer of the tool body shown in phantom lines,

FIG. 4 is a cross section of the tool body taken along section line 4-4 in FIG. 3.

FIG. 5 is the same view as FIG. 3, except only showing the proximal portion of the tool body.

FIG. 6 is an isometric view of the tool body with the outer layer shown in phantom and first and second inner layers removed in a stepped fashion to more clearly indicate the construction of the tool body.

### DETAILED DESCRIPTION

Disclosed herein are delivery tools 10 for delivering an implantable medical lead, wherein the delivery tools include at least one visualization electrode 15 that facilitates the tool being tracked by a mapping system 20 such as, or similar to, one of the St. Jude Medical, Inc. Ensite™ systems. The electrode and conductor configurations employed on the delivery tools 10 result in economical delivery tools 10 that are trackable via mapping systems 20 such as the Ensite™ systems.

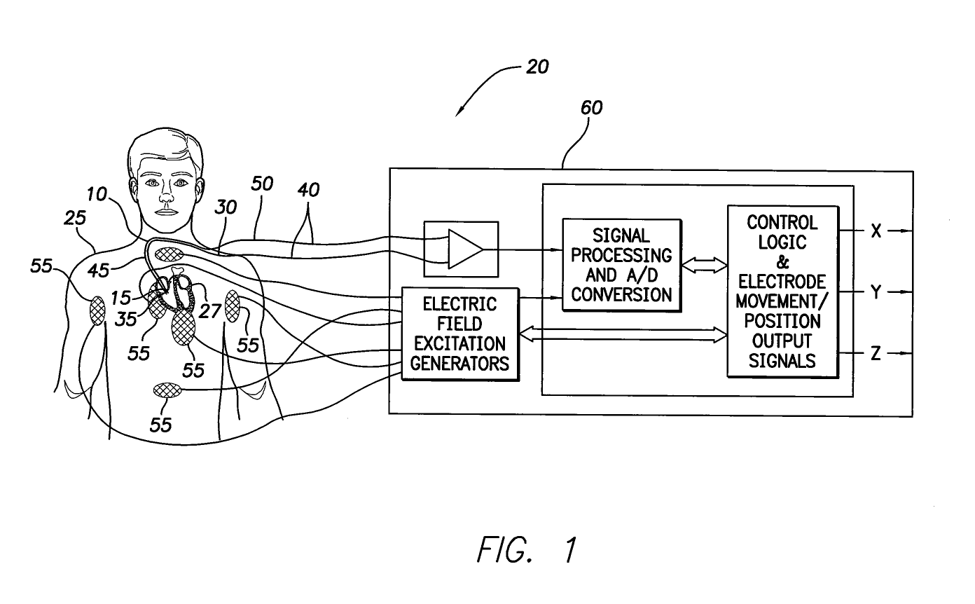

For a general overview of a mapping system 20 similar to an Ensite™ system, reference is made to FIG. 1, which is a diagram illustrating such a mapping system 20 being employed on a patient 25. As indicated in FIG. 1, a delivery tool 10 extends into the right ventricle of a patient's heart 27 via, for example, a subclavian vein access 30 in the patient 25. One or more electrodes 15 are located on the tool 10. For example, in one embodiment, one or more electrodes 15 will be located near the tool distal end 35. One or more conductors 40 extend through the tool tubular body 45 to the tool proximal end 50 to electrically couple with the mapping system 20.

In one embodiment, the mapping system 20 is an Ensite NavX™ imaging and mapping system as marked by St. Jude Medical, Inc. In other embodiments, the mapping systems 20 are other non-fluoroscopy type imaging and mapping systems similar to the Ensite NavX™ system and capable of tracking an electrode of a medical device, such as an electrode equipped lead delivery tool, within a patient. In one embodiment, the mapping system 20 is an imaging and mapping system similar to those disclosed in U.S. Pat. Nos. 5,291,549; 5,553,611; 5,662,108; 6,240,307; 6,939,309; 6,978,168; and 6,990,370, which are incorporated herein by reference in their entireties.

As indicated in FIG. 1, in one embodiment, the mapping system 20 employs three pairs of surface electrodes 55 on the surface of the patient 25. Each surface electrode 55 is electrically coupled to a power and control device 60 that contains the components and logic for operating the system 20 and provides electrical energy to the surface electrodes 55. Each pair of surface electrodes 55 is generally orthogonal relative to the other pairs of surface electrodes 55. The pairs of surface electrodes 55 create generally orthogonal electric fields and are electrically driven via the power and control device 60. The electrical energy to the pairs of surface electrodes 55 is sequenced to allow the potential at the tool electrodes 15 to be sensed for each orthogonal axis. The tool electrodes 15 can be swept over the surface of the chamber (e.g., right atrium, right ventricle, etc.) of the patient's heart 27 to allow the system 20 to generate a three-dimensional (“3-D”) image of the heart chamber. Similarly, the tool electrodes can be swept over the surface of venous anatomy to allow the system to generate a 3-D image of the venous anatomy. The tool electrode movement is tracked by the system 20 and displayed within the 3-D image to allow the physician to visually track the tool 10 within the heart chamber or venous anatomy. The tracking of the tool 10 within the 3-D image allows the physician to much more clearly visualize the tool within the heart chamber or venous anatomy, as compared to fluoroscopy. Additionally, the patient and medical staff are not exposed to fluoroscopy radiation. As a result, lead delivery difficulty is significantly reduced, and patient and medical staff safety is substantially increased.

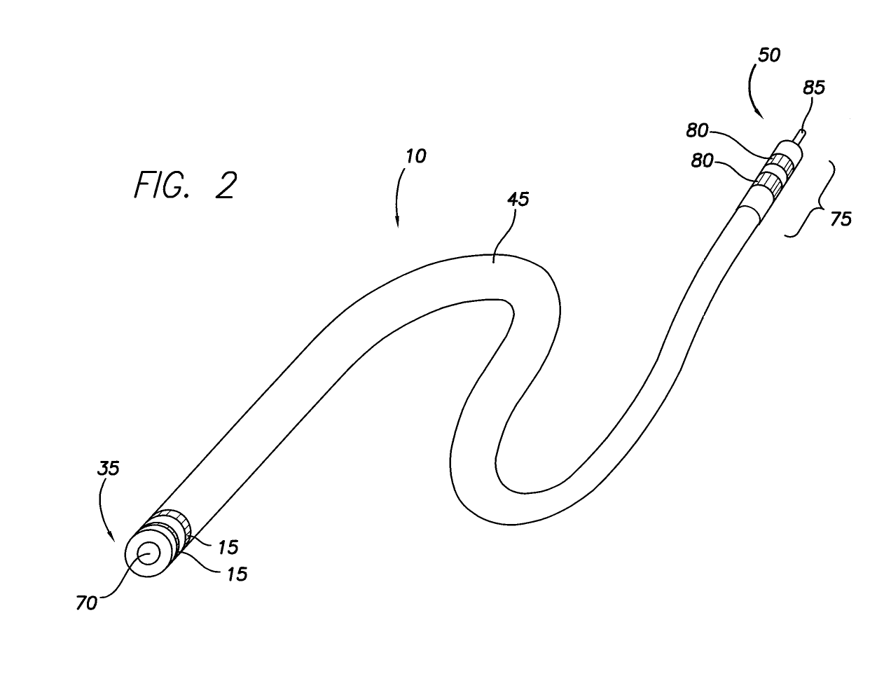

For a discussion regarding a first embodiment of tool 10 for delivering an implantable medical lead, reference is made to FIGS. 2-5. As shown in FIG. 2, which is an isometric view of the tool 10, the tool 10 includes a distal end 35, a proximal end 50, a tubular body 45 extending between the ends 35, 50 and a lumen 70 longitudinally extending through the tubular body between the ends 35, 50. Delivery tools (e.g., catheters, introducers, guidewires, stylets, etc.) and implantable medical leads (e.g., leads employed for pacing, sensing, defibrillation, CRT, etc.) can be passed through the lumen 70 from the proximal end 50 to the distal end 35.

As indicated in FIG. 2, in one embodiment, the electrodes 15 are located on the body 45 near the distal end 35. In other embodiments, the electrodes 15 may be located at other locations on the body 45 in addition to, or besides, the distal end 35, including along significant stretches, if not the entire length, of the body 45. Depending on the embodiment, there will be one, two, three, or more electrodes 15 on the body 15. Each electrode 15 may be electrically independent from the other electrodes 15.

As illustrated in FIG. 1, in one embodiment, the proximal end 50 of the tool 10 will have an adapter or connector assembly 75 configured to facilitate interfacing with the mapping system 20, which may be an Ensite NavX™ imaging and mapping system. The adapter or connector 75 may be similar to an IS-1, DF-1 or IS-4 connector assembly, as long as the adapter or connector 75 allows interfacing with the mapping system 20. For example, the connector assembly 75 may include one or more contact rings 80 and/or a contact pin 85 electrically coupled to respective electrodes 15 via conductors 40 that extend through the body 45. Such a connector assembly 75 may be used to couple the tool 10 to the power and control device 60. Alternatively, the adapter or connector 75 on the tool proximal end 50 may be a 2 mm pin connector to interface with the mapping system 20.

In other embodiments, as discussed later in this detailed description in reference to FIG. 5, the tool proximal end 50 will not have a connector assembly 75. Instead wires 40a, 40b will extend from the tool proximal end 50 to proximally terminate in a connector or adapter, such as a 2 mm pin connector, for interfacing with the mapping system 20, which may be an Ensite NavX™ imaging and mapping.

As shown in FIG. 3, which is a longitudinal elevation of the tool 10 with the electrodes 15 and an outer layer 90 of the tool body 45 shown in phantom lines, a braid layer 95 extends along the tool body 45. In one embodiment, the braid layer 95 includes multiple filars. In one embodiment, the filars will include pair of electrical conductors 40a, 40b helically wound in a first direction along the tool body and a pair of standard braid reinforcement filars or wires 98a, 98b helically wound in a second direction in a second direction opposite from the first direction. In other embodiments, one, two, three or more conductors 40 may be employed, and one, two, three or more reinforcement wires 98 may be employed. In other embodiments, all of the filars of the braid layer 95 will be electrically conductors 40.

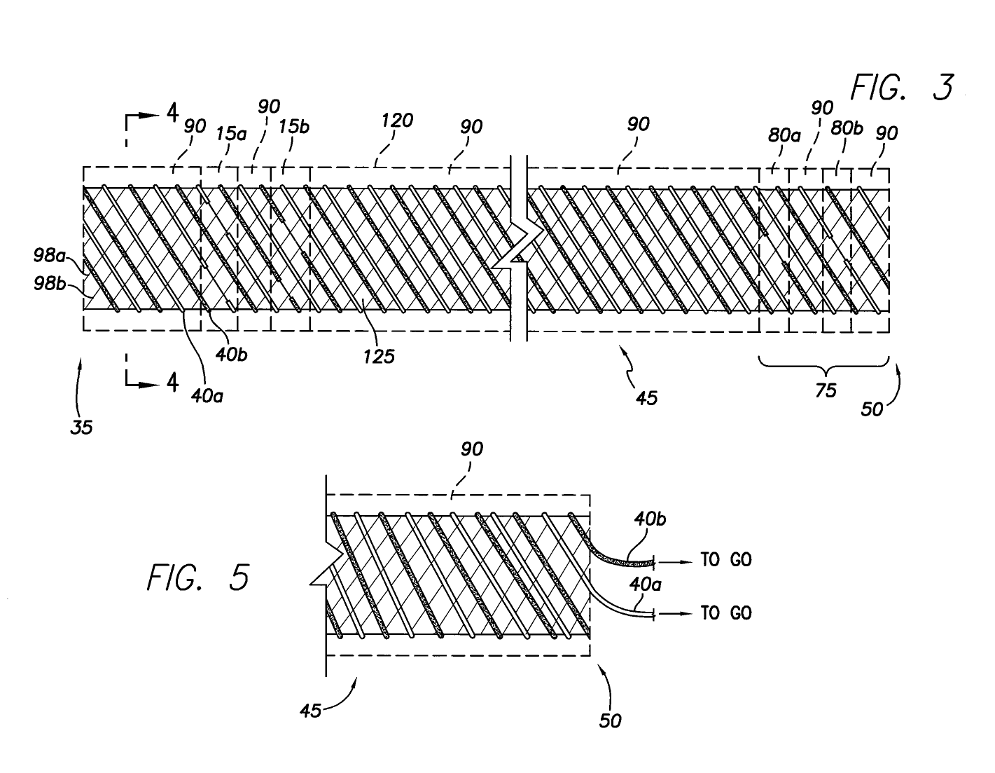

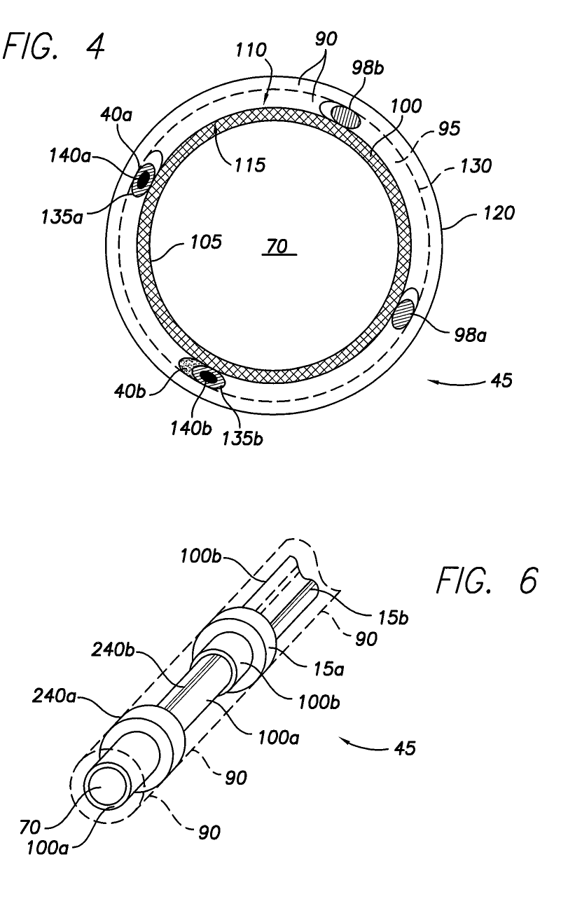

As indicated in FIG. 4, which is a cross section of the tool body 45 taken along section line 4-4 in FIG. 3, the tool body 45 includes an inner layer 100, the braid layer 95, and the outer layer 90. The inner circumferential surface 105 of the inner layer 100 defines the lumen 70, which extends the length of the tool body 45. The braid layer 95 (shown in phantom lines in FIG. 4) extends about an outer circumferential surface 110 of the inner layer 100. With the exception of areas occupied by the electrodes 15 and contact rings 80, as discussed below, the outer layer 90 also extends about the outer circumferential surface 115 of the inner layer 100 such that an inner circumferential surface 115 of the outer layer 90 abuts against the outer circumferential surface 110 of the inner layer 100. The outer circumferential surface 120 of the outer layer 90, in conjunction with the outer circumferential surfaces of the electrodes 15 and contact rings 80, forms the outer circumferential surface 120 of the tool body 45.

As can be understood from FIG. 3, the conductors 40 and reinforcement wires 98 of the braid layer 95 are helically wound spaced from each other, thereby forming spaces or voids 125 in the braid layer 95 between the conductors 40 and reinforcement wires 98. As can be understood from FIGS. 3 and 4, the outer layer 90 extends into the spaces 125 in the braid layer 95 to impregnate the braid layer 95 and bond both the braid layer 95 and outer layer 90 to the outer circumferential surface 110 of the inner layer 100. Thus, as indicated in FIG. 4, the outer boundary 130 (shown in phantom line in FIG. 4) of the braid layer 95 resides near the middle of the thickness of the outer layer 90.

As illustrated in FIG. 4, the conductors 40a, 40b each include an electrical insulation jacket 135a, 135b surrounding a conductive core wire 140a, 140b. In one embodiment, the reinforcement wires 98a, 98b are similarly configured to the jacketed conductors 40a, 40b. However, as indicated in FIG. 4, in one embodiment, the reinforcement wires 98a, 98b are made from a single material to have a uniform cross sectional configuration.

In one embodiment, the conductors 40a, 40b have a diameter of between approximately 0.001″ and approximately 0.025″. In one embodiment, the conductors 40a, 40b have a conductive core 140a, 140b formed of a metal material (e.g., stainless steel, Nitinol, MP35N, copper, silver, gold, etc.) and an electrical insulation jacket 135a, 135b formed of a polymer material (e.g., nylon, polytetrafluoroethylene (“PTFE”), polyimide, etc.).

In one embodiment, the reinforcement wires 98a, 98b have a diameter of between approximately 0.001″ and approximately 0.025″. In one embodiment, the reinforcement wires 98a, 98b have a core formed of a metal material (e.g., stainless steel, Nitinol, MP35N, copper, silver, gold, etc.) and may or may not be insulated with an electrical insulation jacket 135a, 135b formed of a polymer material (e.g., nylon, PTFE, polyimide, etc.). In one embodiment, the reinforcement wires 98a, 98b are formed of carbon fiber or a polymer material (e.g., Dacron, nylon, PTFE, etc.).

In one embodiment, the inner layer 100 has a radial thickness of between approximately 0.001″ and approximately 0.025″, and the inner layer 100 is formed of a polymer material (e.g., “PTFE”, etc.). In one embodiment, the outer layer 90 has a radial thickness of between approximately 0.002″ and approximately 0.010″, and the outer layer 90 is formed of a polymer material (e.g., poly-block amides (“PEBAX”), nylon, silicone rubber, silicone rubber—polyurethane—copolymer (“SPC”), etc.). In one embodiment, the lumen 70 has a diameter of between approximately 0.016″ and approximately 0.099″, and the tool body 45 has an outer diameter of between approximately 0.039″ and approximately 0.122″.

As can be understood from FIG. 3, within the distal and proximal boundaries of the distal electrode 15a and the distal contact ring 80a, the electrical insulation jacket 135a is removed from the first conductor 40a along relatively short segments of the first conductor 40a to place its conductive core 140a into electrical contact with the material forming the distal electrode 135a and distal contact ring 80a. The electrical insulation jacket 135a of the first conductor 40a remains intact throughout the rest of its route along the tool body 45.

As can be understood from FIG. 3, within the distal and proximal boundaries of the proximal electrode 15b and the proximal contact ring 80b, the electrical insulation jacket 135b is removed from the second conductor 40b along relatively short segments of the second conductor 40b to place its conductive core 140b into electrical contact with the material forming the proximal electrode 135b and proximal contact ring 80b. The electrical insulation jacket 135b of the second conductor 40b remains intact throughout the rest of its route along the tool body 45.

In various embodiments, the electrodes 15 and/or contact rings 80 will be formed of metal materials (e.g., platinum-iridium alloy, stainless steel, MP35N, etc.). Such electrodes 15 and/or contact rings 80 will be formed about the braid layer 95 via commonly used methods, and the outer layer 90 will be reflowed about the braid layer 95 between the electrodes and/or contact rings 80 to complete the outer circumferential surface 120 of the tool body 45.

In one embodiment, the electrodes 15 and/or contact rings 80 are formed of a ceramic material loaded with an electrically conductive material. The electrically conductive material of the loaded ceramic material constitutes is of types and in amounts as known in the art to enable a ceramic material to be electrically conductive. The ceramic electrodes and/or contact rings are placed over and adhered to the braid layer (e.g., via an adhesive or brazing). The outer layer 90 is then reflowed about the braid layer 95 between the electrodes and/or contact rings 80 to complete the outer circumferential surface 120 of the tool body 45.

In one embodiment, the electrodes 15 and/or contact rings 80 are formed of a hydrogel material or a polymer material (e.g., PEBAX, silicone rubber, SPC, etc.) loaded with an electrically conductive material (e.g., nickel-coated graphite powder, nickel-coated graphite fibers, etc.). In one embodiment where the loaded polymer material is PEBAX, the electrically conductive material of the loaded PEBAX material constitutes between approximately 10 percent and approximately 50 percent of the total weight of the loaded PEBAX material.

As can be understood from FIGS. 3 and 4, in one embodiment, the braid layer 95 is wound or pulled over the inner layer 100, which is formed of PTFE. Short segments of electrical insulation jacket 135 are removed from the conductors 40 in locations corresponding to the locations of the respective electrode 15 and/or contact ring 80 to be in electrical communication with the conductors 40. The outer layer 90 of PEBAX is then provided about the braid layer 95. In one embodiment, the PEBAX layer 90 is in the form of a tube that is pulled over the braid layer. In another embodiment, the PEBAX layer 90 is sprayed or extruded over the braid layer. Regardless, in one embodiment, the outer layer 90 will have segments that are loaded with nickel-coated graphite powder and positioned to align with the appropriate segments of the conductors 40 having exposed conductive cores 140. The PEBAX layer 90 is then reflowed about the braid layer 95 and PTFE layer 100 to impregnate the braid layer 95 and bond the PEBAX layer 90 to the PTFE layer 100. The PEBAX layer 90 forms the outer circumferential surface 120 of the tool body 45. The loaded segments 15, 80 of the PEBAX layer 90 make electrical contact with the conductive cores 140 of the appropriate conductors 40 such that the loaded PEBAX segments 15, 80 can serve as electrodes 15 and contact rings 80.

As can be understood from FIG. 5, which is the same view as FIG. 3, except only showing the proximal portion of the tool body 45, in one embodiment, the tool 10 will not employ a contact or adapter assembly 75 directly on the tool proximal end 75. Instead, the conductors 40a, 40b will simply extend from the tool body proximal end 50 as free wires that can be coupled to the power and control device 60 of the system 20 via methods known in the art. Alternatively, the free wires will proximally terminate away from the tool proximal end 75 as a contact or adapter assembly employing contact rings 80 or pin connectors, such as a 2 mm pin connector. Such contact or adapter assemblies facilitate the interfacing of tool electrode system with the mapping system 20, which may be an Ensite NavX™ system.

As shown in FIG. 6, which is an isometric view of the tool body 45 with the outer layer 90 shown in phantom and first and second inner layers 100a, 100b removed in a stepped fashion to more clearly indicate the construction of the tool body 45, the tool body 45 can employ conductors 240a, 240b that extend through a layer or on a layer. For example, in one embodiment, the tool body 45 has two inner layers 100a, 100b (a true inner layer 100a and a middle layer 100b extending over the true inner layer 100a), which are surrounded by the outer layer 90. The innermost layer 100a defines a lumen 70 extending through the tool body 45.

As can be understood from FIG. 6, in one embodiment, a first conductor 240a extends along the outer circumferential surface of the inner layer 100a and is covered by the middle layer 100b. A second conductor 240b extends along the outer circumferential surface of the middle layer 100b and is covered by the outer layer 90. The first conductor 240a is in electrical contact with a distal electrode 15a, and the second conductor 240b is in electrical contact with a proximal electrode 15b. In one embodiment, one or both conductors 240a, 240b are formed of electrically conductive inks such as, for example, silver/silver chloride electrode ink or silver/silver chloride/carbone electrode ink, as manufactured by Creative Materials Incorporated of 141 Middlesex Road, Tyngsboro, Mass. 01879.

In one such embodiment, the ink-formed conductors 240a, 240b are deposited on the surfaces of the respective layers 100a, 100b via such methods as screen printing, pad printing, etc. After application of an ink-formed conductor 240a, 240b to its respective substrate, the respective next outer layer is applied over the ink-formed conductor and its respective substrate via such methods as spray deposition, extrusion, reflow, etc., as the case may be. In such embodiments, the electrodes 15a, 15b may be formed of electrically conductive inks in a manner similar to that employed for the ink-formed conductors 240a, 240b, or the electrodes 15a, 15b could be formed of materials similar to those described above with respect to FIGS. 2-4.

In some embodiments, the electrically conductive inks are used to form electrical conductors or traces 240 on the outer circumferential surface of the outer layer 90. An electrical insulation material is then sprayed or otherwise deposited over the ink-formed traces 240 in areas of the traces 240 wherein electrical isolation from the surrounding environment is desired. Electrically conductive inks are used to form electrodes 15 on the outer circumferential surface of the outer layer, and these electrodes 15 are placed in electrical contact with the in-formed traces 240.

As can be understood from FIG. 6, in one embodiment, a first conductor 240a is a longitudinally extending strip of the inner layer 100a. In such an embodiment, the first conductor 240a is formed of the same polymer material as the rest of the inner layer 100a, or is at least compatible with or otherwise joinable to the rest of the inner layer 240a such that the inner layer 100a ends up being an integral whole that includes the first conductor 240a.

Similarly, the second conductor 240b is a longitudinally extending strip of the middle layer 100b. In such an embodiment, the second conductor 240b is formed of the same polymer material as the rest of the middle layer 100b, or is at least compatible with or otherwise joinable to the rest of the middle layer 100b such that the middle layer 100b ends up being an integral whole that includes the second conductor 240b. In such an embodiment, the conductors 240 are formed in their respective layers 100 via such methods as co-extrusion, and the conductors 240 are formed of polymer materials loaded with an electrically conductive material in a manner similar to that discussed above with respect to the electrodes 15 of FIGS. 2-4.

Regardless of whether the conductors 240 are formed of ink or a polymer material loaded with an electrically conductive material, in some embodiments, the conductors 240 are highly flexible, which assists in providing highly flexible tool bodies 45. Additionally, in some embodiments, such conductors 240 do not significantly add to the overall diameter of the tool body 45.

In some of the versions of the above-discussed embodiments depicted in FIGS. 1-6, the electrical connections between the conductors 40, 240 and the corresponding electrodes 15 are made via such methods as brazing, welding, electrically conductive epoxies or adhesives, mechanical crimping or other mechanical methods, etc. In some versions of the above-discussed embodiments, the electrically contacts between the electrodes and conductors may be made via molding the electrode and conductor material together or by simply placing the electrodes and conductors into electrical contact and applying the layers of the body in a manner that maintains the electrodes and conductors in electrical contact.

In some versions of the above-discussed embodiments discussed with respect to FIGS. 1-6, the electrical resistance of the tool 10, as measured from the exterior contact surface of an electrode 15 to the exterior contact surface of its corresponding contact ring 80 is sufficiently low to allow the electrodes to be used for electrogram or pacing or other sensing or treatment functions. However, other versions of the above-discussed embodiments, the electrical resistance of the tool 10, as measured from the exterior contact surface of an electrode 15 to the exterior contact surface of its corresponding contact ring 80 is greater than approximately 100 Ohms.

In one embodiment, the electrical resistance of the tool 10, as measured from the exterior contact surface of an electrode 15 to the exterior contact surface of its corresponding contact ring 80 is at least approximately 200 Ohms. In one embodiment, the electrical resistance of the tool 10, as measured from the exterior contact surface of an electrode 15 to the exterior contact surface of its corresponding contact ring 80 is at least approximately 300 Ohms. In one embodiment, the electrical resistance of the tool 10, as measured from the exterior contact surface of an electrode 15 to the exterior contact surface of its corresponding contact ring 80 is at least approximately 400 Ohms. In one embodiment, the electrical resistance of the tool 10, as measured from the exterior contact surface of an electrode 15 to the exterior contact surface of its corresponding contact ring 80 is at least approximately 500 Ohms. In one embodiment, the electrical resistance of the tool 10, as measured from the exterior contact surface of an electrode 15 to the exterior contact surface of its corresponding contact ring 80 is between approximately 100 Ohms and approximately 6000 Ohms. In one embodiment, the electrical resistance of the tool 10, as measured from the exterior contact surface of an electrode 15 to the exterior contact surface of its corresponding contact ring 80 is between approximately 100 Ohms and approximately 7000 Ohms.

While such high resistances would make an electrode of the tool generally unacceptable for purposes of electrograms or pacing or similar sensing or treatment functions, the high resistance electrodes 15 are adequate for use with a non-fluoroscopy imaging and tracking system (e.g., a St. Jude Medical, Inc. Ensite™ system) to generate cardiac anatomy, potential maps and to track the tool 10.

Where electrode configuration has been optimized for the specific non-fluoroscopy imaging and tracking system, tool electrical resistances exceeding 7000 Ohms can even be useful for purposes of generating cardiac anatomy, potential maps and to track the tool 10.

In one embodiment, as can be understood from FIG. 3, the proximal edge of the distal visualization electrode 15a is spaced apart from the distal edge of the proximal visualization electrode 15b by a distance common for electrodes used for electrograms or pacing, for example, a distance of between approximately 2 mm (a distance common for electrograms) and approximately 11 mm (a distance common for pacing). While such close distances are generally inadequate for electrogram or pacing or similar sensing or treatment functions, the spacing is not so small as to be insufficient for use with a non-fluoroscopy imaging and tracking system (e.g., a St. Jude Medical, Inc. Ensite™ system) to generate cardiac anatomy, potential maps and to track the tool 10. Such close distances between visualization electrodes 15 may facilitate the creation of tools 10 having complicated geometry, extremely tight bend radius, the location of additional features on the tool, etc., than would otherwise be possible with typical electrode spacings used for electrogram or pacing.

In one embodiment, one or more of the electrodes 15 will have a surface area common for electrodes used for electrograms or pacing, for example, a surface area for an individual electrode of between approximately 4.8 mm² and approximately 14.6 mm². While such small surface areas are generally inadequate for electrogram or pacing or similar sensing or treatment functions, the surface area is not so small as to be insufficient for use with a non-fluoroscopy imaging and tracking system (e.g., a St. Jude Medical, Inc. Ensite™ system) to generate cardiac anatomy, potential maps and to track the tool 10. Such small electrode surface areas may facilitate the creation of tools 10 having complicated geometry, extremely tight bend radius, the location of additional features on the tool, increased tool body flexibility, reduced electrode material costs, etc., than would otherwise be possible with typical electrode surface areas used for electrogram or pacing.

While the some of the above-discussed embodiments may have electrodes, contact rings and conductors that result in tools with electrical resistances that are excessively high for electrogram, pacing and similar functions, the embodiments are still advantageous at least because: (1) the tools'electrical resistances are adequate for imaging and tracking purposes when used with a non-fluoroscopy imaging and tracking system (e.g., a St. Jude Medical, Inc. Ensite™ system); and (2) the electrode, contact ring and conductor configurations disclosed herein are inexpensive to manufacture.

Similarly, while the some of the above-discussed embodiments may have electrodes with small spacing and/or small surface areas that make the electrodes inadequate for electrogram, pacing and similar functions, the embodiments are still advantageous at least because the small spacing between electrodes and/or small electrode surface areas: (1) are adequate for imaging and tracking purposes when used with a non-fluoroscopy imaging and tracking system (e.g., a St. Jude Medical, Inc. Ensite™ system); and (2) allow a tool to be constructed with a tighter bending curve and/or greater flexibility; and (3) can result in a less expensive tool to manufacture.

By employing the concepts disclosed in this Detailed Description, visualization electrodes 15 can be economically provided to delivery tools 10 purely for imaging and tracking purposes within a non-fluoroscopy imaging and tracking system (e.g., a St. Jude Medical, Inc. Ensite™ system), thereby enabling such imaging and tracking systems to be used for medical lead implantation and substantially, if not completely, eliminating the need for fluoroscopy during lead implantation.

Although the present invention has been described with reference to preferred embodiments, persons skilled in the art will recognize that changes may be made in form and detail without departing from the spirit and scope of the invention.

What is claimed is:

1. A tool for implanting a medical lead, the tool comprising:
a body including a distal end and a proximal end;
an electrode supported by the body; and
a conductor in electrical contact with the electrode and extending along the body from the electrode to the proximal end, wherein the electrode and conductor form an electrically conductive path that extends from a surface of the electrode to a proximal most point of the conductor on the body, wherein the electrical resistance of the electrically conductive path is at least approximately 100 Ohms.

2. The tool of claim 1, wherein at least one of the electrode and conductor is formed of an electrically conductive ink.

3. The tool of claim 1, wherein at least one of the electrode and conductor is formed of a polymer loaded with an electrically conductive material.

4. The tool of claim 1, wherein the electrical resistance of the electrically conductive path is at least 200 Ohms.

5. The tool of claim 1, wherein the electrical resistance of the electrically conductive path is at least 300 Ohms.

6. A tool for implanting a medical lead, the tool comprising:
a distal end;
a proximal end; a first layer;
a conductor extending along an outer surface of the first layer;
a second layer extending over the outer surface of the first layer; and
an electrode extending over the outer surface of the first layer, forming a portion of the second layer and in electrical contact with an electrically conductive portion of the conductor.

7. The tool of claim 6, wherein the conductor exists in the second layer.

8. The tool of claim 6, wherein the conductor includes an electrically conductive core and an electrical insulation jacket extending about the core.

9. The tool of claim 8, further comprising a braid layer extending over the outer surface of the first layer and the conductor is a helically wound filar in the braid layer.

10. The tool of claim 9, wherein the jacket is missing from at least a portion of a length of the conductor extending through the electrode to place the electrode in electrical communication with the core.

11. The tool of claim 10, further comprising a contact ring extending over the outer surface of the first layer, forming a portion of the second layer and in electrical contact with an electrically conductive portion of the conductor, wherein the jacket is missing from at least a portion of a length of the conductor extending through the contact ring to place the contact ring in electrical communication with the core.

12. The tool of claim 9, wherein the second layer impregnates the braid layer.

13. The tool of claim 6, wherein the electrode is formed of a polymer loaded with an electrically conductive material.

14. The tool of claim 13, wherein the polymer forming the second layer is generally the same as the polymer used to form the electrode.

15. The tool of claim 8, wherein the conductor extends from the proximal end of the tool.

16. A tool for implanting a medical lead, the tool comprising:
a distal end;
a proximal end; a first layer;
a conductor extending along the surface of the first layer; and
an electrode extending over the outer surface of the first layer and in electrical contact with the conductor, wherein at least one of the conductor and electrode is formed of an electrically conductive ink.

17. A system for implanting a medical lead, the system comprising:
an imaging system including a power and imaging device and electrode pairs electrically coupled to the device, wherein the imaging system generates generally orthogonal electrical fields via the electrodes pairs; and
a tool for delivering a medical lead, the tool including a tubular body including a conductor extending from a proximal end of the body to an electrode supported on the body, wherein the conductor is electrically coupled at the proximal end of the body to the device, wherein the electrode is visible via the imaging system and wherein the electrical resistance of an electrically conductive path extending from a surface of the electrode to a proximal most point of the conductor on the body is at least approximately 100 Ohms.

18. The tool of claim 17, wherein at least one of the electrode and conductor is formed of an electrically conductive ink.

19. The tool of claim 17, wherein at least one of the electrode and conductor is formed of a polymer loaded with an electrically conductive material.

20. The tool of claim 17, wherein the tool further includes a braid layer and the conductor is a helically wound filar of the braid layer.

21. A method of delivering an implantable medical lead, the method comprising:
electrically coupling a tool to an imaging system;
generating generally orthogonal electric fields in a patient with the imaging system;
tracking the tool to a lead implantation site, wherein the tool includes an electrode that is visible within the patient via the imaging system, wherein the electrical resistance of an electrically conductive path extending from a surface of the electrode to a proximal most point of the conductor on the body is at least approximately 100 Ohms; and
delivering the lead to the implantation site through the tool.
